(19) United States
(12) Patent Application Publication
(10) Pub. No.: US 2017/0135444 A1
(43) Pub. Date: May 18, 2017

(54) Automated footwear tightening system

(71) Applicant: Martin Gerardo Vincent, Malibu, CA (US)

(72) Inventor: Martin Gerardo Vincent, Malibu, CA (US)

(21) Appl. No.: 15/330,891

(22) Filed: Nov. 14, 2016

Related U.S. Application Data
(60) Provisional application No. 62/254,890, filed on Nov. 13, 2015.

(51) Int. Cl. A43C 11/16; A43C 11/14; H02J 7/32; A61B 5/0205; G08C 17/02; H02J 7/02; A43B 3/00; A61B 5/00

(52) U.S. Cl. CPC A43C 11/165; A43B 3/0015; A43C 11/14; A61B 5/6807; A61B 5/02055; G08C 17/02; H02J 7/025; H02J 7/32

(57) ABSTRACT

An automated footwear tightening system wherein a central rotary closure knob is provided on the top of an article of footwear. The rotary closure knob is engaged with a tightening element embedded within the article of footwear. The rotary closure knob is in communication with a motor, such that the motor can rotate the rotary closure knob to tighten the article of footwear upon a user's foot. A wireless transceiver is in electric communication with the motor. In an embodiment, the wireless transceiver is configured to receive and send wireless signals from a user controlled device and activate the motor. The system allows a user to remotely tighten a shoe upon a wearer's foot via the user's controlled device. In an embodiment, the article of footwear will be provided with sensors to tighten the footwear upon receiving a wearer's foot.

### CROSS-REFERENCE TO RELATED APPLICATION(S)

The present application claims priority to U.S. Provisional Patent Application No. 62/254,890 filed on Nov. 13, 2015, entitled “Digital Tech Shoe Lock via Remote Controlled Mobile App Bluetooth System” the entire disclosure of which is incorporated by reference herein.

### BACKGROUND OF THE INVENTION

1. Field of Invention

The present invention relates to the field of high-tech wearables and the Internet of Things (IoT), and in particular to a remote-controlled tightening system for an article of footwear.

2. Description of Related Art

Archaeological records have dated the use of shoelaces in footwear back to 3500 BC. Shoelaces are considered to be the most common method for fastening footwear onto one's foot. But the use of shoelaces come with a considerable set of draw backs. Lacing of the shoes requires some dexterity. Most young children struggle with learning to tie his/her own shoes. A child's difficult experience with tying his/her own shoes often leads to an accompanying adult needing to complete the task for them. This can be difficult in situations where the adult is looking after more than one child or is in a hurried situation.

The process of shoe tying can also be difficult for adults with limited dexterity. This may include elderly suffering from conditions such as arthritis, which would create increased difficulty in one's ability to tie a proper knot. Limited flexibility may also provide difficulty in tying one's shoes. Injuries could limit one's range of movement and inhibit his/her ability to bend over to tie his/her own shoelaces. Limited flexibility may also occur in the elderly, requiring them to acquire assistance in tying his/her own shoelaces.

Furthermore, shoelaces will often become untied. Repetitive movement may cause the knot of the shoelace to loosen over time, and eventually this can lead to the lace becoming completely untied. An untied shoelace will cause the shoe loosen, which may cause the shoe to be accidently removed from the user's foot. An untied shoe lace can also present a potential hazard as it may become caught on another object, stepped on by another person, or the user may end up tripping over his/her own untied shoelace.

Several designs have attempted to replace laces. A “slip-on” shoe is designed without laces. In place of shoelaces, an elastic material is provided so that the opening of the shoe may be stretched to better receive a user's foot. But with no tightening mechanism provided, a user may not be able to engage in strenuous activity. During such activity, the shoe may become removed from his/her foot. Furthermore, the elastic material may wear out after some duration, causing an even looser fit.

Velcro, or hook and loop fasteners, have also been implemented in footwear to replace laces. Hook and loop straps can provide an adequate tightening system to replace shoelaces, but the long term effects of fastening and unfastening the hook and loop straps may cause failure. Hook and loop straps tend to wear over time, and may completely lose their ability to fasten. User's must also be flexible enough to reach down and fasten the hook and loop straps.

Buckles are another alternative to shoelaces and are commonly found on footwear. Buckles require limited dexterity, but still require a user to reach down to fasten them. Furthermore, buckle systems employ manufacturer determined holes for the buckles to fasten with. The spacing of the holes may not provide the necessary fine adjustment for the user to tighten his/her footwear appropriately. Users are often left with an adjustment that is either too tight or too loose.

In recent decades, a shoe tightening system utilizing a central rotary closure has been developed. Such a tightening system is presented in U.S. Pat. No. 5,502,902, in which a central rotary closure provides a knob to be rotated by users to tighten or loosen a shoe about their foot. The central closure is further constructed to allow the user to engage a quick release by pulling or pushing the knob. While the central rotary closure provides a great alternative to shoelaces, it still requires the user to reach down to engage the knob.

Based on the foregoing, there is a need in the art for a shoe tightening system which a user can easily adjust without reaching down. Furthermore, what may be desired is a system in which a user can remotely tighten or loosen his/her own shoes or the shoes of others, weather the wearer is stationary or actively in motion (such as walking, jogging, running, etc.)

### SUMMARY OF THE INVENTION

The objective of the present invention is to create an automated system to tighten an article of footwear upon a user's foot via sensors or a remote controlled device. The automated footwear tightening system comprises of an article of footwear provided with a central rotary closure on the top surface of the article of footwear. The central rotary closure knob is engaged with a tightening element. A motor is provided to rotate the central closure knob to tighten or loosen the engaged tightening element. The motor communicates electically with a wireless transceiver (a transmitter and receiver combined in one) to allow a user to wirelessly signal the motor to tighten or loosen the article of footwear by rotating the rotary closure knob automatically.

In a preferred embodiment, the rotary closure knob is further provided with a quick release to disengage the rotary knob from the tightening element. In an embodiment, the rotary closure knob will be provided on top of an instep cover. The instep cover will help distribute pressure to the top of a wearer's foot.

In an embodiment, the wireless transceiver will be further provided with a port for charging and/or sharing data. Additionally, the wireless transceiver may be provided with one or more electronic contact buttons to allow a user to power the system on or off, or to allow a user to engage in wireless pairing. In an embodiment, the wireless receiver is configured to send and receive Bluetooth radio signals.

In an embodiment, the present invention is further provided with an induction charging system to charge the wireless receiver. In another embodiment, the automated footwear tightening system is provided with one or more sensors to track movement, location, heart rate, blood pressure, weight, distance, and body temperature. In an embodiment, the present invention is provided with one or more kinetic energy generators to provide charge to the system gathered from movement of the wearer.

In another embodiment, the automated footwear tightening system is provided with a piezo-electric sensor. In an embodiment, the piezo-electric sensor is configured to charge the system, and in another embodiment, the piezo-electric sensor is configured to activate the motor to loosen or tighten the article of footwear upon a wearer's foot.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a left side elevational view of the automated shoe, according to an embodiment of the present invention;

FIG. 2 is a front elevational view of the automated shoe, according to an embodiment of the present invention;

FIG. 3 is a top plan view of the automated shoe, according to an embodiment of the present invention; and

FIG. 4 is a flowchart depicted a method of operation for the automated shoe tightening system, according to an embodiment of the present invention;

### DETAILED DESCRIPTION OF PREFERRED EMBODIMENTS

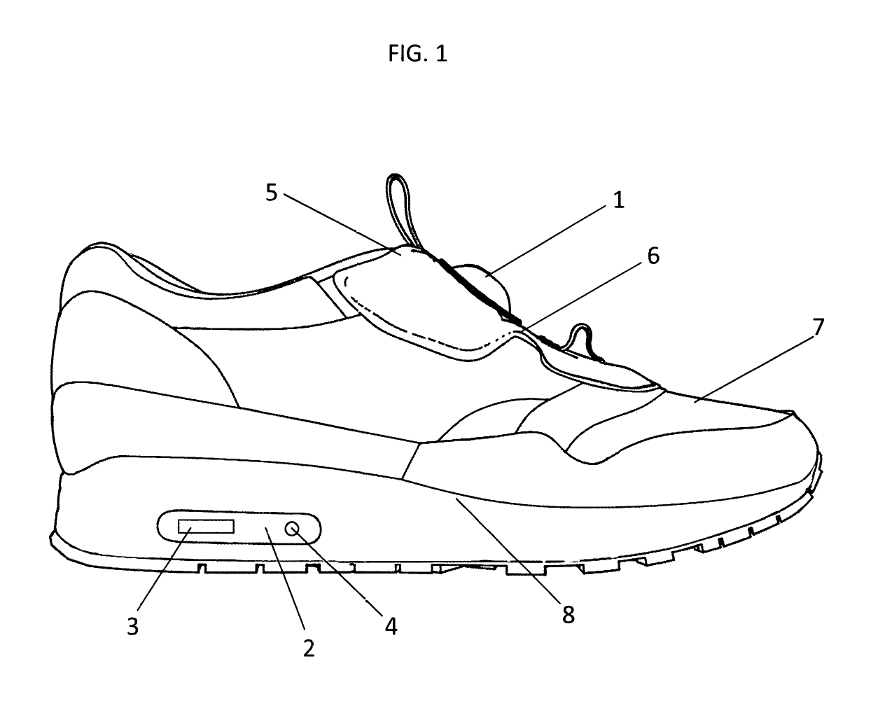

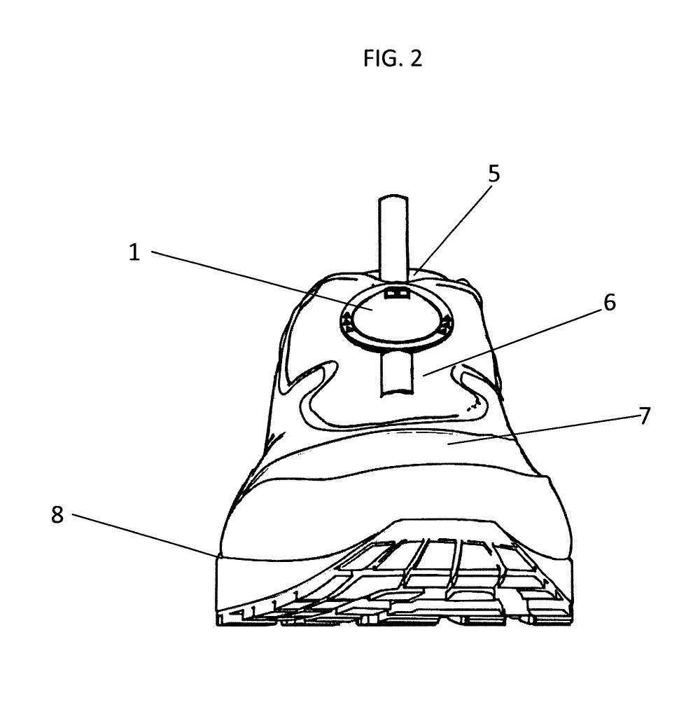

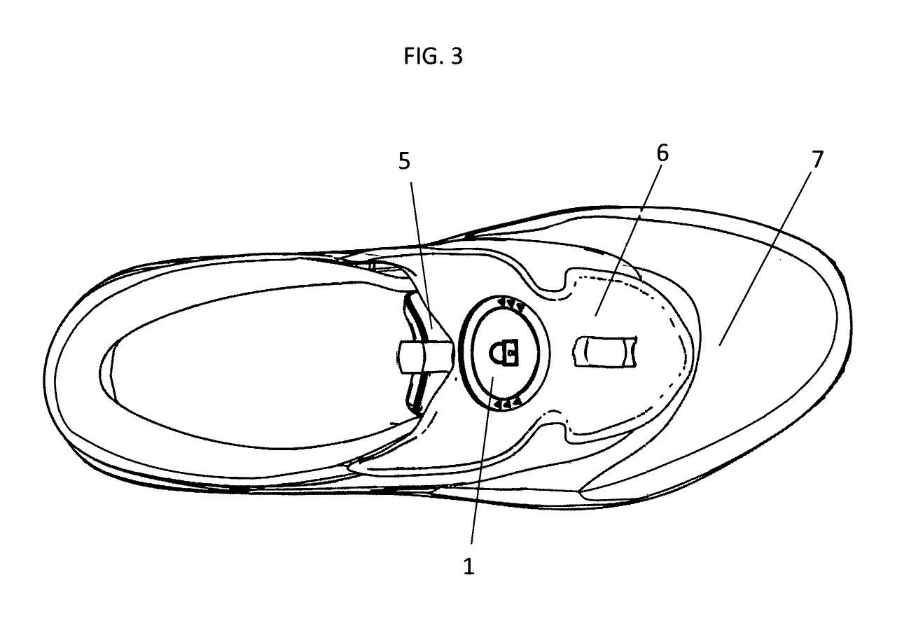

Preferred embodiments of the present invention and their advantages may be understood by referring to FIGS. 1-4 wherein like reference numerals refer to like elements.

In reference to FIG. 1-3, an embodiment of the present invention is shown as an article of footwear, more specifically a shoe as a high-tech wearable. Preferably, an athletic or leisure shoe. Consisting of soft, flexible tongue 5 configured to receive a user's foot. An instep cover 6 is integrated on the top of the tongue 5. In an embodiment, the instep cover 6 is sewn on to the tongue 5. In another embodiment, the instep cover 6 may be adhered onto the tongue 5, or secured by another means of attachement as deemed appropriate by one skilled in the art. The instep cover 6 is further provided with a central rotary closure knob 1 (the type used on the shoes of the mentioned patent). The central rotary closure knob 1 engages with a tightening element (not shown). In an embodiment, the tightening element is a metal or plastic wire or rope with a relatively small diameter. In another embodiment, the tightening element is a strap. The tightening element is embedded within the upper portion 7 of the shoe. The tightening element passes through one or more hooks (not shown) also embedded within the upper portion 7 of the shoe, and anchored at the top of the sole 8, as is known in the art.

In an embodiment, a user may rotate the closure knob 1 in a specified direction to wrap the tightening element around a center post (not shown). As the tightening element is wrapped, tension between the hooks and tightening element is increased. If a user's foot is provided within the shoe, the tension will tighten the instep cover 6 down onto the user's foot, as is known in the art of the mentioned patent. In an embodiment, the user may press a button or lift the knob to quickly release the tightening element from the center post and loosen the shoe.

In a preferred embodiment, the closure knob 1 is rotatable by an electric motor provided within the central rotary closure 1. In an embodiment, the electric motor configured such that a user may still operate the closure knob 1 manually. The electric motor communicates electrically with a wireless transceiver 2, via one or more wires (not shown) embedded within the upper portion 7 and sole 8 of the shoe. In the preferred embodiment, the wireless transceiver 2 is provided within the sole 8 of the shoe. In an embodiment, the wireless transceiver 2, is further provided with one or more buttons 4. The buttons 4 may be used to activate a connection link (e.g., Bluetooth, IR, radio, or similar wireless connection types). In another embodiment, one button is a power button provided to activate the wireless transceiver. In another embodiment, one button activates both power and a wireless connection link. In an embodiment, the shoe will automatically enter a sleep state when left untouched for a specified duration of time, and the shoe will power on when movement is picked up by threshold detection of a sensor, such as an accelerometer.

In a preferred embodiment, the wireless transceiver 2 is further provided with a port 3. The port 3 is adapted to receive a connection to charge a power source or rechargeable battery provided within the wireless transceiver. In a preferred embodiment, the rechargeable battery is a 5-volt, lithium ion battery. In another embodiment, the battery may be any voltage or type deemed appropriate by one skilled in the art. In another embodiment, the port 3 is a wired serial port, such as a USB port. The wired port will be configured to charge the battery contained within the wireless transceiver 2. Additionally, the wired port will be able to connect to a computer or electronic device to allow the user access to additional settings and configuration options.

In a preferred embodiment, the high-tech wearable shoe is placed on an inductive charging mat to recharge the battery provided within the wireless transceiver 2 of the shoe. The charge is wirelessly transmitted through electro-magnetic induction of coils provided within the mat and the shoe, as is known in the art. In another embodiment, the shoe is provided with a coil placed towards the bottom of the sole 8 to allow for more efficient charging.

In a preferred embodiment, the automated shoe is to be paired with a software application to be loaded onto a user's mobile device. In an embodiment, the user downloads the appropriate software application from his/her preferred application store. Upon opening up the application, the user is prompted to set up the software, allowing the application access to features of the mobile device and pair the mobile device with the shoes. In a preferred embodiment, once the application is properly configured, the user is presented with an interface allowing them to activate the motor housed within the shoe. The user may interact with buttons presented on the screen of his/her mobile device, so they user may press the buttons to engage the motor to tighten or loosen remotely. In a preferred embodiment, the user chooses a tightness level numerically ordered from zero to nine, where nine is the tightest setting and zero is the loosest setting, wherein the tightening element has not been engaged to wrap around the center post of the rotary closure unit. In another embodiment, the numerical scale may be any range or scale deemed appropriate to provide enough adjustment for the shoe to fit precisely on a user's foot. In another embodiment, the level of tightness may be activated by a slider on the screen of the mobile device. In another embodiment, the level of tightness may be adjusted via voice command, as known in the art. In another embodiment, the screen of the mobile device may display a knob for the user to rotate to adjust the level of tightness. In another embodiment, any other visual representation of an adjustable scale may be used to allow the user to adjust the tightness of the shoes on a mobile device.

In a preferred embodiment, the software application also presents the user with the charging and connectivity status of the shoe. In addition, the software may allow a user to track his/her movement, calories burned, and/or calculate the total distance traveled. Using the software application, a parent could track the location of his/her children who are wearing the automated shoes. In another embodiment, the software may also display information such as altitude change, height jumped, average speed, or other information which a user may consider useful. In an embodiment, the software application is further provided with a voice command feature. The voice command feature should allow a user to command the with accepted commands. In an embodiment, if a user says “tighten” the shoe will tighten by one unit of tightness specified by the application. If a user says “loosen” the shoe will loosen by one unit of tightness specified by the application. If a user says “release” the shoe will completely loosen. If a user says “track” the application will begin to track his/her movement data. In an embodiment, other voice commands may be added as deemed functional by one skilled in the art.

In an embodiment, the present invention is further provided with an integrated circuit board embedded within the wireless transceiver 2. The circuit board may contain components and/or sensors such gyroscopes, accelerometers, and GPS receiver chips. In the embodiment, the circuit board is configured to track the movements of the user, and wirelessly communicate gathered information to the user's mobile device. In another embodiment, bio-sensing elements will be integrated into the footwear to provide data as to the wearer's heart rate, blood pressure, weight, calories burned, and body temperature. In another embodiment, the shoe may be equipped with piezo-electric technology and circuitry to allow the battery provided to be charged from the pressure of the user's steps and weight.

In another embodiment, one or more kinetic energy generators are provided to charge the provided battery. Wherein each kinetic energy generator comprises of a weight with a semi-circular shape, attached to a first gear. The first gear is attached to a second gear which is connected to an electric coil. The arrangement is provided, such that, upon the kinetic movement of a wearer, the weight rotates due to gravity, rotating the first gear, driving the second gear, and inducing a current into the electric coil. The electric coil is in communication with the battery, and the current is transmitted to the batter to be stored.

In an embodiment, within the software application, a user can select his/her shoe type as athletic. This will allow for an automated tightening feature, wherein the shoe automatically tightens when the sensors embedded in the shoe relay data attributed to running. When the user slows down to walking speed, the shoe will automatically loosen a bit for more comfort as the sensors detect the slower, less intense movement. In an embodiment, the automated tightening feature can be set by the user to change the degree to which the shoe is tightened when running begins.

In an embodiment, a user will be able to save and select his/her preferred tightness settings from a memory bank. In an embodiment, wherein an athletic shoe type is paired, the user will also be able to store his/her preferred running tightness level for the shoe. In an embodiment, if a user owns more than one pair of automated tightening shoes, the user will be able to store his/her preferred tightness setting for each pair of automated tightening shoes within the software application.

In another embodiment of the present invention, the rotary closure knob is further equipped with a torsion or pressure sensor. In an embodiment, the sensor will limit the tightness level as to not over tighten the shoe onto a user's foot. In another embodiment, the sensor will set the scale for a user to choose various tightness levels according to his or her needs.

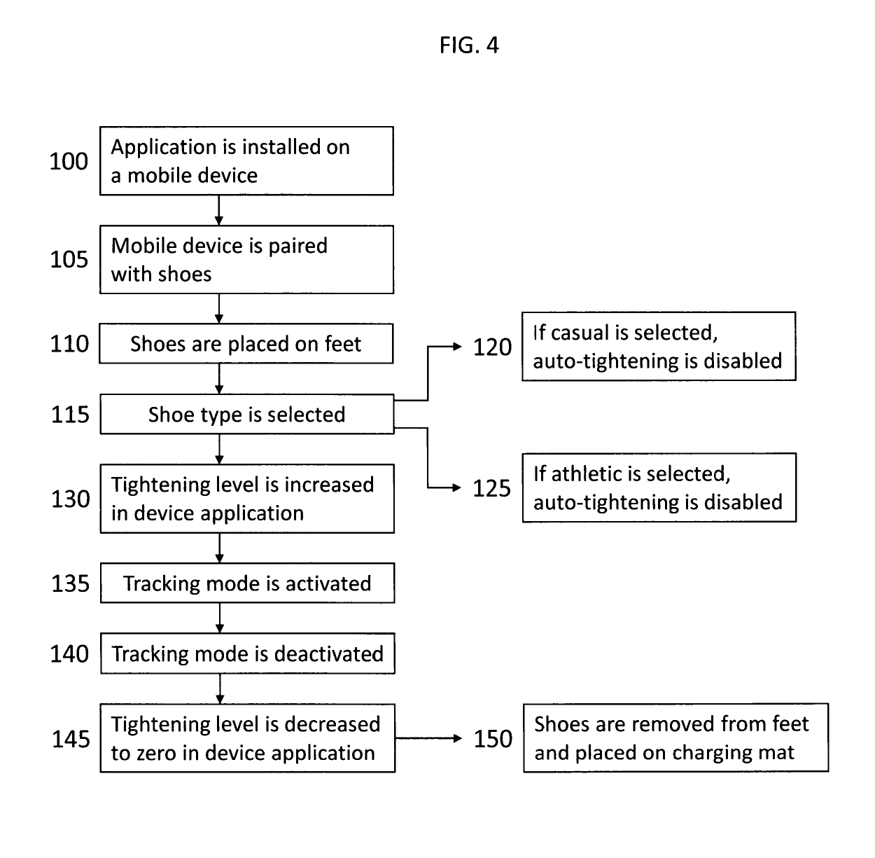

In reference to FIG. 4, a flowchart provides a visual representation for a method of use to remotely control the automated shoe tightening system using a mobile device application. In a preferred embodiment, at step 100, the software application is downloaded, installed, and configured onto a mobile device. Installation and configuration may require a user to accept terms of agreement and allow the application to access some hardware of the mobile device, such as the microphone, display, speaker, or other components of the mobile device. In an embodiment, the application is downloaded from a preferred application store such as the iTunes, Google Play, Windows Apps, or another online store in which applications may be downloaded.

In an embodiment, a user may control one or more pairs of shoes simultaneously within the application of the paired mobile device. This feature would allow a parent to control the tightness of his/her own shoes, and his/her children's shoes.

In a preferred embodiment, at step 105, the shoes are paired with the mobile device, preferably via Bluetooth. In the preferred embodiment, the shoes are paired through the application, wherein the application has access to the mobile device's Bluetooth hardware and configuration. In another embodiment, the pairing is accomplished though the Bluetooth configuration provided by the mobile device. In another embodiment, the pairing is achieved through IR, radio, or another means of wireless device communication. In an embodiment, a button is provided to activate the shoes and enable them to receive a wireless connection. In another embodiment, the shoes are automatically activated when the embedded sensors receive a threshold signal or the shoes are removed from a charging device, such as a cable or mat.

In a preferred embodiment, at step 110, the shoes are placed onto to the feet of a user. This may be the feet of the user in control of the mobile device, or an individual who will have the tightness of his/her shoes controlled by a user with the paired mobile device. If the rotary closure knob has been tightened, it must first be set back to zero either manually via the knob, or through the application on the paired mobile device. In a preferred embodiment, at step 115, the user selects the shoe type on the application of his/her mobile device. In a preferred embodiment, the user may select between an athletic or casual shoe type setting. In another embodiment, the user may select between two or more shoe types deemed appropriate for the application. In a preferred embodiment, at step 120, the casual shoe type is selected by the user. The selection of a casual shoe type will disable the auto-tightening feature, such that the tightness level of the shoe will remain constant whether the user is sitting, walking, or running. In a preferred embodiment, at step 125, the athletic shoe type is selected. The selection of an athletic shoe type will enable the auto-tightening feature, such that the tightness level of the shoe will increase to a preferred setting when the sensors embedded within the shoe determine that the user is running or jogging.

In a preferred embodiment, at step 130, the tightening level is increased by a user via the application loaded on the paired mobile device. The tightening level may be increased via user input on a displayed scale, button, knob, or slider. In an embodiment, the user may select a preferred level of tightness that is stored within the software application of the paired mobile device. In another embodiment, when the user's foot is received by the shoe and the user applies weight onto that foot, the pressure sensors within the shoe will activate the closure knob to tighten to the user's stored preferred level of tightness.

In a preferred embodiment, at step 130, a tracking mode is activated within the software application of the paired mobile device. When activated, the tracking mode may track the movements, calories burned, location, altitude, jumped height, or other information deemed useful for the wearer of the automated tightening shoes. In a preferred embodiment, at step 135, the tracking mode is deactivated. Deactivation of the tracking mode ceases data collection, compiles the gathered data, and presents it to the user via a display in the software application. In an embodiment, the tracking mode is automatically activated when movement is sensed by the sensors embedded within the automated shoes. In another embodiment, the tracking mode is automatically deactivated when the sensors embedded in the automated shoes do not register movement for a specified duration of time.

In a preferred embodiment, at step 145, the tightness level of the shoes is decreased such that no tension is applied to the wearer's foot. In an embodiment, the tightness level is decreased to zero by a slider, knob, button, or displayed scale. In an embodiment, the tightness level may be manual decreased to zero via a button or pull knob provided as part of the rotary closure mechanism. In a preferred embodiment, at step 150, the wearer removes the shoes at a tightness level of zero and places them onto a charging mat. The batteries provided within the shoes are then charged via electro-magnetic induction, as is known in the art. In an embodiment, the user presses a button on the shoe to deactivate it. In another embodiment, the shoe is automatically deactivated when it is set to charge or when no movement is registered for a specified duration of time.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. An automated footwear tightening system comprising:
a. an article of footwear;
b. a central rotary closure knob provided on the top surface of the article of footwear;
c. a tightening element engaged with the central rotary closure knob;
d. a motor in communication with the central rotary closure knob; and
e. a wireless transceiver in electronic communication with the motor; wherein the motor is activated by the wireless transceiver to rotate the central rotary closure knob, winding the tightening element, and tightening the article of footwear.

2. The automated footwear tightening system of claim 1, wherein the central rotary closure further comprising with a quick release feature.

3. The automated footwear tightening system of claim 1, further comprising an instep cover beneath the central rotary closure knob.

4. The automated footwear tightening system of claim 1, wherein the wireless transceiver further comprises:
a. a port for charging and sharing data; and
b. one or more electronic contact buttons able to be pressed by a user.

5. The automated footwear tightening system of claim 1, wherein the wireless transceiver sends and receives Bluetooth radio signals,

6. The automated footwear tightening system of claim 1, wherein the wireless transceiver is adapted to be charged through induction.

7. The automated footwear tightening system of claim 1, wherein the wireless transceiver further comprises one or more sensors to track movement.

8. The automated footwear tightening system of claim 1, wherein the article of footwear further comprising one or more kinetic energy generators.

9. The automated footwear tightening system of claim 8, wherein one or more kinetic energy generators provide charge to the wireless transceiver.

10. The automated footwear tightening system of claim 1, wherein the article of footwear further comprising a piezo-electric sensor.

11. The automated footwear tightening system of claim 10, wherein the piezo-electric sensor provides charge to the wireless transceiver.

12. The automated footwear tightening system of claim 10, wherein the piezo-electric sensor activates the motor.

13. A method of wearing the automated footwear tightening system comprising:
a. installing a software application onto a user controlled device;
b. pairing the user controlled device with an automated pair of footwear;
c. placing the automated pair of footwear on a wearer's feet; and
d. using the software application on the paired device to tighten the automated pair of footwear onto the wearer's feet.

14. The method of wearing the automated footwear tightening system of claim 13, further comprising selecting an auto-tightening feature on the user controlled device.

15. The method of wearing the automated footwear tightening system of claim 13, further comprising selecting a tracking feature on the user controlled device.

16. The method of wearing the automated footwear tightening system of claim 13, further comprising a voice command feature on the user controlled device.
